(19) United States
(12) Patent Application Publication
(10) Pub. No.: US 2021/0368087 A1
(43) Pub. Date: Nov. 25, 2021

(54) CAMERA EXPOSURE CONTROL WHEN ACQUIRING FLUORESCENT IN SITU HYBRIDIZATION IMAGES

(71) Applicant: LEICA BIOSYSTEMS IMAGING, INC., Vista, CA (US)

(72) Inventor: Karl RATCLIFF, Newcastle upon Tyne (GB)

(21) Appl. No.: 17/281,534

(22) PCT Filed: Oct. 23, 2019
(86) PCT No.: PCT/US2019/057717

§ 371 Date: Mar. 30, 2021

(30) Foreign Application Priority Data
Oct. 24, 2018 (EP) 18202387.9

(51) Int. Cl. H04N 5/235; G06T 7/00; G06T 5/20

(52) U.S. Cl. CPC H04N 5/2353; G06T 7/0012; G06T 5/20; G06T 2207/10056; G06T 2207/20036; G06T 2207/10064; G06T 2207/30024; H04N 5/2351

(57) ABSTRACT

A fluorescence in situ hybridization, FISH, image acquisition apparatus and a method for controlling a FISH image acquisition apparatus. A FISH probe image is acquired (S24, S44). Image processing using morphological operations is applied to identify areas of interest around locations of elevated image intensity. From this, a mask is generated (S22, S42) which filters out all but the areas of interest. The mask is applied to determine image intensity in the areas of interest. If the image intensity does not have a desired value (S26), indicating incorrect exposure of the image, then the method is repeated with one or more different exposures until an adequately exposed FISH probe image is obtained (S29, S47, S48). The correct exposure is determined either empirically by iteratively applying stepwise increases (S28), or analytically by applying a formula (S46).

### FIELD OF THE INVENTION

The present disclosure relates generally to fluorescent in situ hybridization (FISH) and more specifically to methods and apparatus for acquiring FISH images.

### BACKGROUND

FISH is a widely used cytogenetic technique based on attaching fluorescent probes to chromosomes to detect, analyze and quantify nuclear abnormalities through detecting the presence of specific sequences of deoxyribonucleic acid (DNA) or ribonucleic acid (RNA). There are many different implementations of FISH, a few of which are: (a) tissue FISH; (b) multi-spectral FISH (M-FISH); (c) spot counting FISH; (d) flow-fish (i.e. FISH carried out in a flow cytometer); and (e) microfluidics-assisted FISH (MA-FISH). Applications of FISH include diagnosis of several cancer types, analysis of the progression of several cancer types, and identification of chromosomic abnormalities. Often, as well as acquiring a FISH probe image, i.e. an image that is sensitive to the fluorescent label used to tag the DNA or RNA of interest, a counterstain image is also acquired using a stain such as DAPI (4′,6-diamidino-2-phenylindole) to stain the nuclear DNA and hence identify the locations of nuclei in the sample. The FISH probe image and the counterstain image can then be stored together and processed jointly, e.g. for analysis or display.

The following patent publications in the name of Applied Imaging Corporation (now part of Leica Biosystems, Inc.) describe methods and apparatus for acquiring FISH images: U.S. Pat. Nos. 5,880,473 A, 6,225,636 B1, US 2002/081014 A1, US 2003/012420 A1, and WO 98/52018 A1, the full contents of which are incorporated herein by reference.

WO 2008/019299 A2 discloses an auto-exposure method for capturing acceptably exposed images from all regions of a sample. The method specifically addresses the situation in which the dynamic range of the image sensor and associated electronics is less than the range of intensities of the different regions in the sample, so no single captured image is capable of storing all the signal information. To solve this problem, multiple images are taken. Namely, a first image is taken with a correct exposure for the brightest image areas. However, since areas with dimmer signal will be underexposed in the first image, and perhaps not recorded at all in view of the signal lying outside the dynamic range, a second image is taken at a greater exposure to image these dimmer areas, while at the same time masking the bright areas. The process can be iterated to take further images with successively decreasing exposure as desired until the set of images as a whole includes correctly exposed image portions over the whole sample area.

EP 3021105 A1 discloses another auto-exposure method for capturing acceptably exposed images from all regions of a sample which also addresses the same problem that, when the dynamic range of the image sensor and associated electronics is less than the range of intensities of the different regions in the sample, significant signal from the dim regions may be lost. The method is based on using a pre-defined set of exposure times, e.g. 0.1 seconds, 1 second, 4 seconds and 40 seconds. A first image is acquired with one of these exposure times, which is either the best exposure for the whole image, or an exposure time in the middle of the set. The first image is then processed region-by-region to determine an intensity value for each region. If the intensity value for a particular region is below the detector's saturation value and above the detector's background noise level, then it is determined what the intensity value would be for the longer exposure times in the set. The software then selects the particular exposure time in the set which is predicted to give the highest intensity value that is below saturation. A further image is then taken at that exposure value and the sub-image “tile” covering that particular region is then the one that is used in subsequent image processing. The image ultimately used for the post-processing of the image is thus a composite mosaic of sub-image tiles with different exposures, each tile having the exposure that has been selected for that region.

Known microscopes and other systems that acquire FISH probe images set the exposure of the camera using an auto-exposure technique that relies on image histograms, or other image measurements such as minimum and maximum pixel values, to determine the brightness, i.e. image intensity, of the FISH probe image. The histograms for determining an appropriate exposure are created from the whole FISH probe image or from a selected portion of the FISH probe image. In the latter case, it is known to create the histograms from a portion of the image such as the central portion, or by dividing the whole image into zones and taking a consensus reading from individual brightness values determined for each zone, or by identifying where the FISH signals are located by applying an image threshold and from that creating a mask to mask out areas with below-threshold intensity, so that only the areas with above-threshold intensity are taken account of for the exposure determination. By way of example, a known microscope system with an auto-exposure function for acquiring FISH probe images is CytoVision® supplied by Leica Biosystems, Inc.

However, it has been observed that FISH probe images acquired with standard auto-exposure setting methods as just described sometimes lack detail. In particular, on closer investigation, it seems that in some cases the portions of the image of interest where the FISH probes are located are not optimally exposed, i.e. either underexposed or overexposed.

### SUMMARY OF THE INVENTION

According to one aspect of the disclosure, there is provided a method of controlling an image acquisition apparatus comprising a fluorescence microscope arrangement to acquire a fluorescence in situ hybridization, FISH, probe image of a sample labeled with a FISH probe. The method comprises:

setting an exposure for acquiring a FISH probe image to an initial value;

acquiring a FISH probe image with the set value of the exposure;

image processing the FISH probe image by applying at least one morphological operator to identify areas of interest around locations of elevated image intensity, and then generating a FISH probe image mask for filtering out all but the areas of interest;

applying the mask to the FISH probe image to determine image intensity in the areas of interest; and,

if that image intensity has a desired intensity value which indicates correct exposure of the FISH probe image, then generating and saving a FISH record including a correctly exposed FISH probe image, else, adjusting the exposure value and iterating the method with the adjusted value of the exposure.

The mask is used to image process the FISH probe image to determine image intensity within the mask regions, in particular at the cell nuclei. The adjusted value of the exposure for the final image that is saved in the record is then determined based on the image intensities within the mask regions.

In one group of embodiments, the adjusted exposure is determined empirically by successive increases in the exposure until the image intensity is above a desired value, so that the desired value acts as a threshold that needs to be exceeded. Namely, the initial value of the exposure is intentionally set to be too short, so that the initial FISH probe image will be underexposed. By starting the acquisitions with the first FISH probe image being underexposed, the adjusted value of the exposure for the next acquisition will be greater than the initial value. With an initial, intentionally underexposed FISH probe image, the signal in the nuclear regions will almost certainly be too low. From the image intensity in the areas of interest in the underexposed FISH probe image, a higher exposure can be determined which provides sufficiently bright imaging of the FISH signal. In particular, the exposure can be iteratively increased from its initial value, so as to eventually arrive at an exposure which captures a FISH probe image that is sufficiently bright. However, in the case that there are no features, e.g. nuclei, or insufficiently few, which have been fluorescently tagged by FISH, then there can be no signal, or insufficient signal, in the FISH probe image, so the cell areas (i.e. the areas under the mask) will never fluoresce, or not fluoresce strongly enough, and so remain dark, or too dim, regardless of the exposure. Since it cannot be known a priori whether there will be any, or sufficient, FISH signal, the incrementing of the exposure is preferably terminated after the iterations result in a maximum exposure value having been reached or approached. This measure avoids unnecessarily repeating the iterative acquisition of FISH probe images when there is no prospect of finding a sufficiently strong FISH signal.

In particular, in this group of embodiments, determining the adjusted value of the exposure comprises acquiring subsequent FISH probe images with exposures successively increased from the initial value and image processing them to determine their image intensities until an exposure is arrived at that provides an image intensity in the areas of interest that is above the desired image intensity. Moreover, as discussed above, it can be useful to terminate the incremental increase of the exposure once a maximum exposure value is reached without obtaining a FISH probe image with an above-threshold image intensity. In this case, the FISH record is generated and saved with one of the FISH probe images with below-threshold image intensity, such as the most recently acquired one, and with metadata, such as a text label in a header of the record, indicating that the FISH probe image most likely has no or insufficient FISH signal.

In theory at least, the inverse of the proposed method based on an underexposed initial FISH probe image would be possible. That is the first FISH probe image could be acquired with intentional overexposure, and the exposure then decremented until a suitable image intensity was reached. However, that would not be as good, since the method would start with the longest exposures, e.g. several seconds, and therefore would take orders of magnitude longer to execute compared with the claimed method, where the initial exposure duration will typically be perhaps a few milliseconds, e.g. 5 ms.

In another group of embodiments, the adjusted exposure is determined analytically by applying a formula to calculate what exposure is required for the image intensity to reach a desired value based on the image intensity, and exposure, of the initially acquired FISH probe image. The desired image intensity thus acts as a target value that is aimed for in this group of embodiments. For example, the adjusted value of the exposure can be determined by multiplying a first factor based on the ratio between the exposure and the image intensity of the initial FISH probe image with a second factor based on the desired image intensity. In other embodiments, determining the adjusted value of the exposure comprises acquiring at least one subsequent FISH probe image, each subsequent FISH probe image having an exposure successively increased starting from the initial value, and further comprising: image processing the subsequent FISH probe images to determine their image intensities; and determining the adjusted value of the exposure either: by multiplying a first factor based on a sum of the ratios of the exposures and the image intensities of a plurality of the initial and subsequent FISH probe images with a second factor based on the desired image intensity; or by multiplying a first factor based on the ratio between the exposure and the image intensity of one of the subsequent FISH probe images with a second factor based on the desired image intensity.

Optionally, the sample may additionally be labeled with a counterstain, in which case the method may further comprise acquiring a counterstain image of the sample. The counterstain image is then image processed to generate a counterstain image mask that filters out all but areas of, or around, the counterstained areas. The subsequent image processing of the FISH probe image can then be confined to the areas not filtered out by the counterstain image mask, i.e. the FISH probe image processing only takes place on areas of the sample that are counterstained, or areas that are closely related to the counterstained areas, e.g. if a dilation operator is applied to the counterstained pixels for generating the counterstain mask. Applying a dilation operator in this way may be beneficial to help avoid any registration errors between the counterstain image and the FISH probe images causing clipping by the counterstain mask of areas in the FISH probe image where there is FISH signal. With a typical counterstain used for FISH, the counterstained feature locations will be cell nucleus locations, since the counterstain will attach to the nuclear material in the sample.

A further aspect of the invention provides a computer program product for controlling an image acquisition apparatus to acquire a fluorescence in situ hybridization, FISH, probe image of a sample labeled with a FISH probe and optionally also a counterstain, the computer program product bearing machine readable instructions for performing the above described methods of controlling an image acquisition apparatus.

A still further aspect of the invention relates to an image acquisition apparatus for acquiring a fluorescence in situ hybridization, FISH, probe image of a sample labeled with a FISH probe. The image acquisition apparatus comprises a fluorescence microscope arrangement operable to acquire FISH probe images of a sample; and a control computer configured to determine appropriate exposures for acquiring FISH probe images with the fluorescence microscope arrangement. The control computer determines an appropriate exposure by:

setting an exposure for acquiring a FISH probe image to an initial value;

acquiring a FISH probe image with the set value of the exposure;

image processing the FISH probe image by applying at least one morphological operator to identify areas of interest around locations of elevated image intensity, and then generating a FISH probe image mask for filtering out all but the areas of interest;

applying the mask to the FISH probe image to determine image intensity in the areas of interest; and,

if that image intensity has a desired intensity value which indicates correct exposure of the FISH probe image, then generating and saving a FISH record including a correctly exposed FISH probe image, else, adjusting the exposure value and iterating the method with the adjusted value of the exposure.

The apparatus may further comprise: a display; and a display output operable to access the FISH record and transmit the FISH probe image, and optionally the counterstain image, to the display such that the FISH probe image, and optionally also the counterstain image, are displayed.

A still further aspect of the invention relates to a clinical network comprising:

a computer;

a data repository configured to store FISH records, the FISH records including FISH probe images, and optionally counterstain images;

network connections enabling transfer of the FISH records or parts thereof between the computer and the data repository; and

an image acquisition apparatus according to the above operable to generate FISH records and save them to the data repository.

### BRIEF DESCRIPTION OF THE DRAWINGS

In the following, the present invention will further be described by way of example only with reference to exemplary embodiments illustrated in the figures.

FIG. 1 is a schematic drawing of an example fluorescence microscope system suitable for acquiring FISH images.

FIG. 2 is a flow diagram showing steps involved in performing a method according to an embodiment of the invention.

FIG. 3A shows a FISH probe image which was taken without benefit of the invention.

FIG. 3B shows a FISH probe image of the same sample as FIG. 3A taken with an optimized exposure according to an embodiment of the invention.

FIG. 4 is a flow diagram showing steps involved in performing a method according to an alternative embodiment.

FIG. 5 shows an example computer network which can be used in conjunction with embodiments of the invention.

FIG. 6 is a block diagram of a computing apparatus that may be used for carrying out the image processing needed to implement the invention.

### DETAILED DESCRIPTION OF THE PREFERRED EMBODIMENTS

In the following detailed description, for purposes of explanation and not limitation, specific details are set forth in order to provide a better understanding of the present disclosure. It will be apparent to one skilled in the art that the present disclosure may be practiced in other embodiments that depart from these specific details.

In this document we use the term morphological operator. This term is understood to mean a mathematical operator used for shape analysis, and in particular for extracting image components that are useful in the representation and description of shape. For FISH probe images, this means the shape of cells or cell elements, such as nuclei. Example morphological operators are: dilation, erosion, opening, closing. Further detail can be found in:
Chapter 9, entitled “Morphological Image Processing”, of the textbook Gonzalez and Woods “Digital Image Processing” 3rd edition (2008), pp. 627 to 688, ISBN 013168728, and
Chapter 14, entitled “Edge Detection in Microscope Images”, of the textbook “Image Analysis in Histology” Edited by Richard Wootton, David Springall and Julia Polak (1995), pp. 241-261, ISBN 0521434823,
   the full contents of which are incorporated herein by reference.

### Microscope Platform

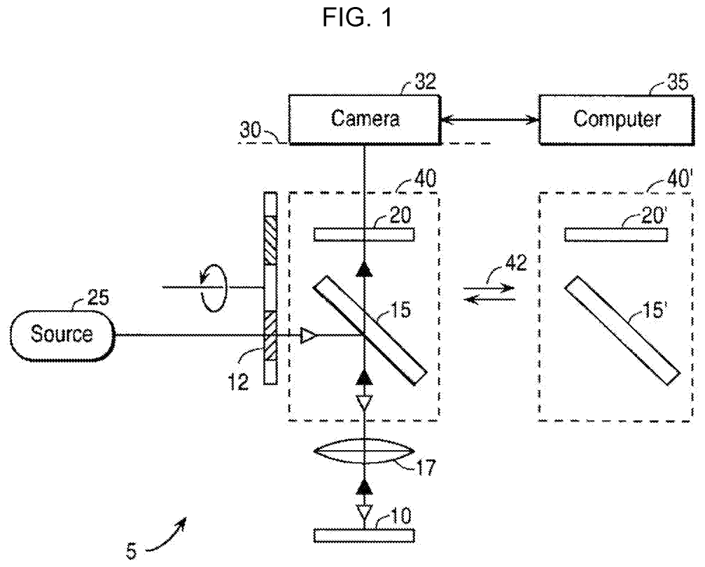

FIG. 1 is a schematic drawing of an example reflection-mode (i.e. epi) fluorescence microscope system 5 that is suitable for acquiring FISH images of a sample 10. The optical arrangement in the microscope system 5 includes an excitation filter 12 (shown as one of several such filters on a filter wheel), a dichroic mirror 15, a microscope objective 17 (say 60× to 100× image capture magnification), and an emission filter 20 (sometimes also referred to as a barrier filter). Excitation light from a source 25 passes through the excitation filter 12, is in part reflected by the dichroic mirror 15 and that part proceeds through the microscope objective 17 to the sample 10. The excitation light traveling towards the sample is shown schematically by hollow arrowheads. Fluorescent radiation emitted from the sample 10 passes back through the objective 17, through the dichroic mirror 15, and through the emission filter 20 to form an image in an image plane 30. The fluorescent light traveling away from the sample is shown schematically by solid black arrowheads. The image is digitized by a camera 32, such as one having a charged-coupled device detector, and the digitized image is sent to a computer 35 for subsequent processing.

If the filters have single passbands, the particular filters and dichroic mirror are specific to a single dye in the sample. Images for other dyes in the sample are acquired by substituting optical elements configured for the excitation and emission bands for each other dye. The dichroic mirror and the emission filter are typically rigidly mounted to a supporting structure 40 (shown in phantom), often referred to as a cube, with multiple cubes being movable into and out of the optical path. Oppositely directed arrows 42 represent a suitable mechanism such as a rotatable turret or a detented slide mechanism. The multiple excitation filters are typically deployed on a rotatable filter wheel (as shown). The system may be for monochrome image acquisition or color image acquisition. For color acquisition the CCD camera is a color CCD camera, multiband excitation is provided in three color bands with respective sources, or a broadband source and appropriate filters, and three corresponding emission filters are provided that only transmit within one of three emission bands. Example fluorescent dyes that are used for FISH include: DAPI, FITC (fluorescein isothiocyanate) and cyanine dyes such as Cy3, Cy3.5, Cy5, Cy5.5, and Cy7.

### Image Acquisition

The image acquisition of the FISH probe images includes applying a camera auto-exposure method. The auto-exposure method is conceived to set the camera exposure to a suitable value for obtaining a FISH probe image so that the FISH signals are clear and visible, provided that the sample is capable of producing a FISH signal of sufficient brightness. In particular, the proposed method is robust against the presence of bright, highly fluorescent debris which would otherwise result in underexposure of the cell nucleus areas where the FISH fluorescence signal is located.

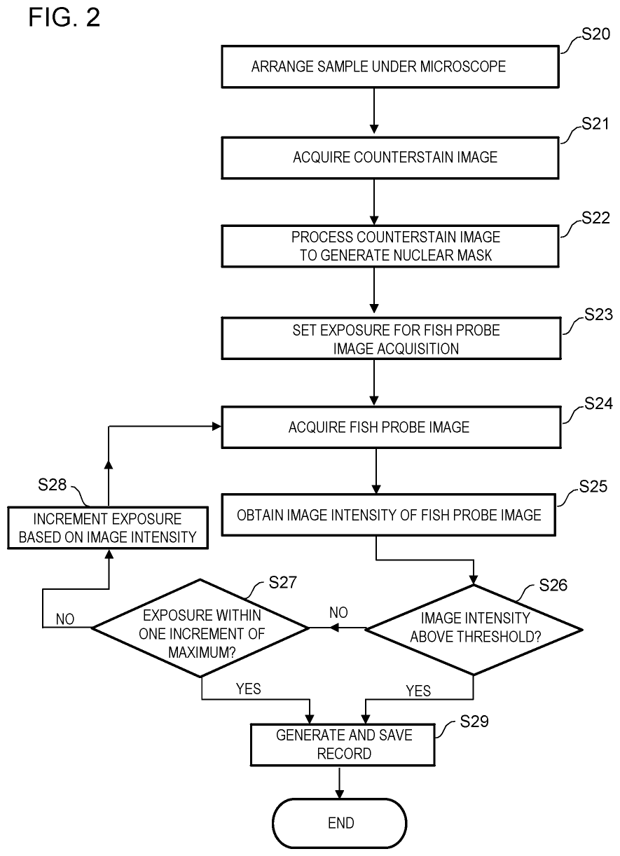

FIG. 2 is a flow diagram showing steps involved in performing a method embodying the proposed auto-exposure method.

First, as shown in Step S20 it is of course necessary to provide a sample, e.g. on a slide. The sample has been labeled with a FISH probe and a suitable counterstain. The sample is loaded under the microscope, or other suitable apparatus platform with microscope imaging capability such as a suitably equipped flow cytometer or microfluidic system. The counterstain typically highlights the cell nuclei, but in principle the counterstain could additionally or instead stain other cell features such as cytoplasm or cytoplasmic membrane. In the following we assume a nuclear counterstain.

In Step S21, the method acquires a counterstain image of the sample and saves it.

In Step S22, the method image processes the counterstain image to generate a mask defining areas of interest, i.e. the areas of the nuclei. This may be done, e.g. by applying an image intensity threshold, to produce a mask identifying the locations of the cell nuclei. For example, a closed contour can be determined around each contiguous group of pixels identified to correspond to a nucleus. The closed contours could be used directly for the mask, or the contours could be further processed, e.g. by dilation. Another alternative would be to use some kind of blob analysis to define the mask. Some amount of dilation or other expansion of the nuclear areas may be advantageous to allow for registration errors between the counterstain image and the FISH probe images which are subsequently analyzed using the mask. Namely, if the mask exactly corresponds to the stained nuclear pixels in the counterstain image, then any registration error will result in some proportion of the FISH signal being excluded from the determination of image intensity, and hence make the measured image intensity lower than the true image intensity.

In Step S23, the method sets the exposure of the image acquisition apparatus to an initial value which is expected to result in an underexposure of the FISH probe image.

In Step S24, the method acquires a FISH probe image.

In Step S25, the method image processes the FISH probe image by applying at least one morphological operator to identify areas of interest around locations of elevated image intensity, and then generating a FISH probe image mask around the areas of interest, so that the mask filters out all but the areas of interest. The counterstain mask generated in Step S22 assists the image processing in Step S25 by allowing the image processing to be confined to the areas of interest as defined by the counterstain mask, thereby reducing the amount of processing required and avoiding processing of artefacts not lying in areas of cell material of interest.

### Example Computation for Step S25 (Fish Probe Image Mask Generation)

a. The FISH probe image is filtered by the counterstain mask to set the intensity value of all pixels to zero that are masked out by the counterstain mask.
b. A median filter is applied for noise reduction.
c. A white top hat filter is applied to remove any background ambient light component of the FISH signal preserving image intensity features that are brighter than the background and objects that are smaller than a certain size, wherein the size is chosen so as not to reject any features that are likely to occur with the current image capture magnification (or with a range of likely image capture magnifications).
d. A regional maximum operator is applied to determine the locations of maxima within the FISH probe image. A regional maxima can be defined as a contiguous set of connected pixels, each pixel having an intensity value above a threshold, whose external boundary pixels all have a value less than the threshold.
e. The regional maxima are dilated by applying a dilation operator to avoid the mask filtering out too much.
f. The FISH probe image mask is generated by applying a threshold filter to the image, with the threshold being set so that pixels with above-threshold intensities correspond to the locations of FISH signals.

In Step S26 to S28, the method adjusts the exposure as necessary based on the image intensity in the areas of interest. If the image intensity measurements indicate that brightness in the areas of interest is below a threshold value, i.e. too dark, the method increments the exposure to E′=E+ΔE unless the increased exposure value would exceed a maximum permitted value (test of Step S27). If the maximum exposure is not exceeded, the process flow proceeds from Step S27 to Step S28 and then further to Step S24 and so on to obtain another FISH probe image with the increased exposure. If the maximum exposure would be exceeded by the increment, as tested for in Step S27, this indicates that there is no or insufficient FISH signal to measure, since the longest permitted exposure has still resulted in a FISH probe image which is too dark at the positions where the nuclei are located, in which case the method can end. On the other hand, if the intensity measurements indicate the brightness is high enough, then the exposure was OK, and the iterative part of the method can end after generating and saving a record in Step S29.

In other words, Step S26 tests whether the image intensity obtained in Step S25 has a desired intensity value, which indicates correct exposure of the FISH probe image. If ‘yes’, then the method can terminate by generating and saving a FISH record including a correctly exposed FISH probe image. It is also sensible to save the counterstain image into the record, although not essential. If ‘no’ then it is tested in Step S27 whether it is sensible to increment the exposure value.

In Step S27, if the incremented exposure value would be higher than a maximum exposure value, then this means in all probability that the sample is not capable of emitting a sufficiently strong FISH signal, so there is no point in iterating further. The method is therefore terminated by saving a record containing, for example, the most recently acquired FISH image. In this case it is also sensible, although not essential, to add some metadata, such as a text label in the record header, stating or indicating that the FISH probe image most likely has no significant or insufficient FISH signal, i.e. this is most likely a failed acquisition.

If Step S27 permits the exposure to be incremented, then this is done in Step S28, and the process flow returns to Step S24 to acquire a further FISH probe image.

It is envisaged that adjusting the exposure to find an appropriate value will comprise repeatedly acquiring FISH probe images with incrementally increasing exposures by repeated traversal of the loop of Steps S24 to S28, with image processing determining at each iteration the FISH image intensity at the areas of interest, e.g. cell nuclei, until an exposure is arrived at that provides an image intensity which is sufficiently bright (test of Step S26), i.e. above a lower image intensity threshold.

The exposure increments, may for example be constant fixed increments, or increments whose size varies. One example of a varying increment would be to follow a predefined progression such as increasing by a multiplying factor, e.g. 2 to double the exposure each time. Preferred values for the multiplying factor in these embodiments is between 1.5 and 2.5. Another example would be to vary the increment size interactively based on how far below the desired intensity level the last FISH probe image was determined to be, with the increments becoming smaller as the desired intensity level is approached, e.g. by using a multiplying factor that includes as a component the difference between the desired intensity and the current intensity.

With Step S29, the method concludes by saving a FISH probe image into a record. The preferable outcome is when the flow proceeds from Step S26 to Step S29, in which case the FISH probe image that is saved has an intensity value in the areas of interest that is of a desired value. The record preferably also saves the counterstain image. The FISH probe image that is saved may be the one most recently acquired, including the ‘nul’ dark image (in case of flow from Step S27 to Step S29) in the case the maximum exposure limit has been reached. Alternatively, the saved image might be an additional FISH probe image acquired as part of Step S29. Making an extra acquisition for the record may be to obtain an overall better quality image. For example, an exposure might be chosen that is deemed to be optimum based on a joint analysis of two or more of the FISH probe images acquired through different traversals of Step S24, e.g. an exposure value determined by interpolation of the image intensities of the most recent two or three iterations of the loop of Steps S24 to S28.

It is preferable if the processing of the images during the above acquisition process is done rapidly so that the method is acceptably quick, e.g. with the aid of a graphics processing unit (GPU). Moreover, the exposure increment ΔE is preferably set to a value that is small enough to provide fine adjustment, but not so small that the number of iterations makes the process as a whole unacceptably slow. In addition, it is noted that speed is the reason why the method starts with short exposures and increases them rather than the opposite, i.e. starting with long exposures and then shortening them.

The mask generated from the FISH probe image and the mask generated from the counterstain image may be generated using any combination of standard processing techniques, for example as described in the above-referenced textbook chapters. The FISH probe images or the counterstain images may be color or grayscale. The images may be modified by applying a contrast enhancement filter. The mask generation may involve some segmentation to identify the areas of interest in the image. Segmentation may involve any or all of the following image processing techniques:

1. Variance based analysis to identify the seed areas

2. Adaptive thresholding

3. Morphological operations (as described above)

4. Contour identification

5. Contour merging based on proximity heuristic rules

6. Calculation of invariant image moments

7. Edge extraction (e.g. Sobel edge detection)

8. Curvature flow filtering

9. Superpixel clustering.

These image processing steps for generating the masks are described by way of example and should not be interpreted as being in any way limitative on the scope of the invention.

### Comparative Examples

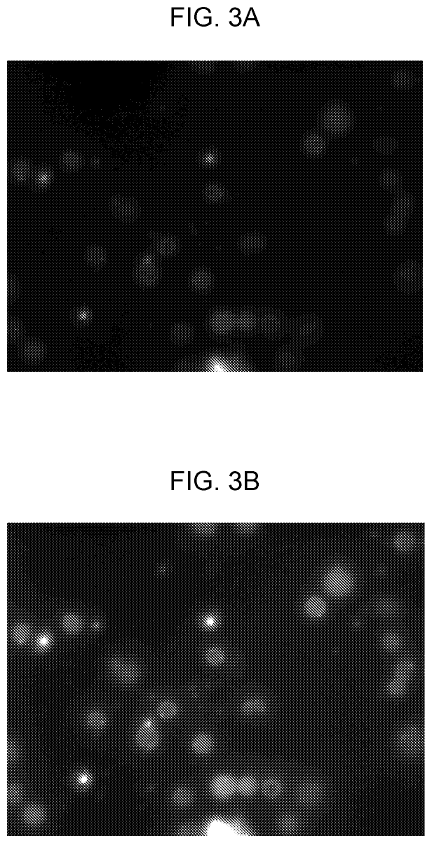

FIG. 3A shows a FISH probe image taken without benefit of the invention. The bright fluorescent debris that is visible near the bottom edge of the image centrally from left to right has caused the auto-exposure method based on brightness across the whole image to underexpose the areas of interest, so they are too dark.

FIG. 3B shows a FISH probe image of the same sample as FIG. 3A taken with an optimized exposure according to an embodiment of the invention. The mask obtained from the FISH probe image has been used to exclude the brightly fluorescing debris from influencing the exposure, which is determined as described above. As a result, the areas of interest in the FISH probe image are appropriately exposed and the detail in the cell nuclei is visible.

### Alternative Image Acquisition Embodiments

As an alternative to the above-described empirical approach of initially deliberately underexposing and then making stepwise increases in the exposure until the image intensity is above a threshold, it is possible instead to determine what should be a suitable exposure analytically by calculating what exposure is required for the image intensity to reach a desired image intensity in the areas of interest in the final FISH probe image. Such a calculation may be based on scaling the exposure used for the initial image up or down through comparing the image intensity of the initial FISH probe image with the desired image intensity for the final FISH probe image. The exposure for the final FISH probe image can thus be set directly from the image intensity determined from the initially acquired initial FISH probe image without iteration. Namely, if the image intensity in a given FISH probe image is a certain fraction, e.g. in one example a third, of the desired image intensity, then the correct exposure can be inferred to be the inverse of the fraction, i.e. 3 times in our example, the exposure used to obtain that FISH probe image. Namely, the adjusted value of the exposure, E_{final}, can be determined by multiplying a first factor based on the ratio between the exposure used to acquire the initial image, E₀, and the image intensity of the initial FISH probe image, I₀, with a second factor based on the image intensity that is desired, Iₜₕ, that is:

$${E}_{final} = {I}_{th} \frac{{E}_{0}}{{I}_{0}}$$

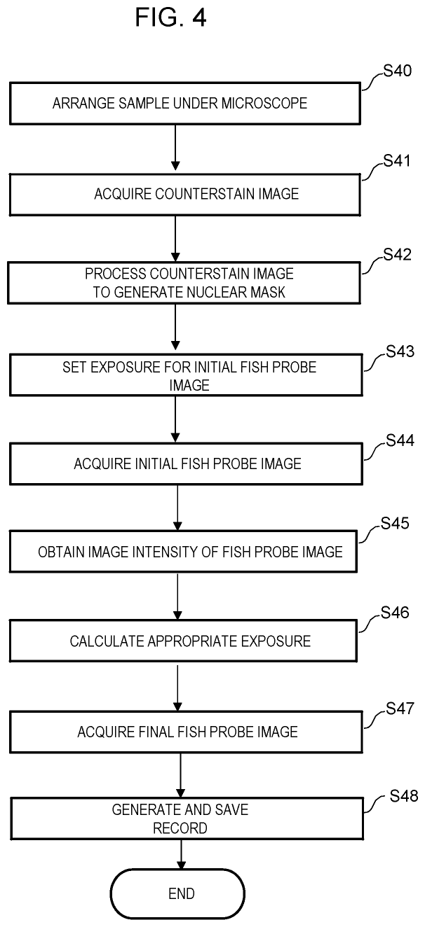

FIG. 4 is a flow diagram showing steps involved in performing a method embodying such an auto-exposure method.

Step S40 corresponds to step S20 of FIG. 2. This step provides a sample that has been labeled with a FISH probe and a suitable counterstain and loads the sample into the acquisition apparatus.

In Step S41, which corresponds to step S21 of FIG. 2, the method acquires a counterstain image of the sample and saves it.

In Step S42, which corresponds to step S22 of FIG. 2, the method image processes the counterstain image to generate a mask defining areas of interest, i.e. the areas of the nuclei.

In Step S43, which corresponds to step S23 of FIG. 2, the method sets the exposure of the image acquisition apparatus to an initial value, which may, for example, either one that is expected to generate an underexposed FISH probe image or one that is expected to be correctly exposed.

In Step S44, which corresponds to step S24 of FIG. 2, the method acquires a FISH probe image.

In Step S45, which corresponds to step S25 of FIG. 2, the method image processes the FISH probe image by applying at least one morphological operator to identify areas of interest around locations of elevated image intensity, and then generates a FISH probe image mask for filtering out all but the areas of interest. This mask is then applied to the FISH probe image to determine image intensity in the areas of interest.

In Step S46 an appropriate exposure is calculated according to the formula:

$${E}_{final} = {I}_{th} \frac{{E}_{0}}{{I}_{0}}$$

In Step S47, the final FISH probe image is acquired with the exposure calculated in Step S46.

In Step S48, which corresponds to Step S29 of FIG. 2, a FISH record is generated into which is saved the final FISH probe image and preferably also the counterstain image. Analogous to the embodiment of FIG. 2, if desired, the image intensity of the final FISH probe image acquired in Step S47 can be determined by applying the same measures as for Step S45, and if the image intensity is too low (or too high) compared with a desired value, as measured by being above a threshold or within a desired range between lower and upper limits, then the record can have metadata saved into it to indicate that the FISH signal is too weak (or too strong).

It will be appreciated that Steps S40 to S45 are essentially the same as Steps S20 to S25 respectively, other than in the analytical approach of FIG. 4 unlike that of FIG. 2 there is no necessity that the initial FISH image has its exposure chosen to deliberately underexpose the image. This is not necessary in the analytical method, since the formula that is applied will operate equally well with underexposure or overexposure of the initial image.

A hybrid approach between the embodiments of FIG. 2 and FIG. 4 could also be used in which two or more FISH probe images are acquired with different exposures, e.g. by iteratively incrementing the exposure two or more times, and then interpolating between the image intensity values from these images to determine an appropriate exposure for the final acquisition. An example of such an analytic way of determining an appropriate final value for the FISH probe image exposure comprises acquiring at least one subsequent FISH probe image following the flow of FIG. 2, so that, including the initial FISH probe image, at least two FISH probe images are acquired, from which the exposure for the final FISH probe image is calculated. It is envisaged that the subsequent FISH probe image or images have exposures that are successively increased from the previous one, starting from an increment to the initial value for the first subsequent FISH probe image. The final exposure value, E_{final}, is determined by multiplying a first factor based on a sum of the ratios of the exposures and the image intensities of the initial and subsequent FISH probe images with a second factor based on the image intensity desired, e.g. according to the formula:

$${E}_{final} = {I}_{th} {{{\sum }^{N}}_{n - 0}}_{{I}_{n}}^{{E}_{n}}$$

where

n=0 corresponds to the initial FISH probe image,

n≥1 corresponds to the or each subsequent FISH probe image, and

N is the number of subsequent FISH probe images, where N≥1.

This could be modified if desired by only taking some of the elements in the summation, e.g. discarding one or more of the iterations relating to the lowest ‘n’ numbers. Another modification would be to weight the sum elements in some sensible way, such as giving the most recently acquired subsequent images a higher weighting, where the weighted formula would be:

$${E}_{final} = {I}_{th} {\sum }_{n - 0}^{N} {{c}_{n}}_{{I}_{n}}^{{E}_{n}}$$

where cₙ are the weighting factors.

Another alternative would be only to use one of the subsequent FISH probe images in the determination, i.e.:

$${E}_{final} = {I}_{th} \frac{{E}_{n}}{{I}_{n}} , where 1 \leq n \leq N$$

that is multiplying a first factor based on the ratio between the exposure and the image intensity of one of the subsequent FISH probe images with a second factor based on the image intensity desired.

Further variations on this theme are readily appreciable.

### Computing Platforms and Network Environment

The proposed image processing may be carried out on a variety of computing architectures, in particular ones that are optimized for image processing, which may be based on central processing units (CPUs), GPUs, field-programmable gate arrays (FPGAs) and/or application-specific integrated circuits (ASICs). In some embodiments, the image processing software runs on Nvidia GPUs from Nvidia Corporation, Santa Clara, Calif., such as the Tesla K80 GPU. In other embodiments, the image processing software can run on generic CPUs. Faster processing may be obtainable by a purpose-designed processor for performing image processing calculations.

It will be understood that the computing power used for running the image processing software, whether it be based on CPUs, GPUs or some other processor type, may be hosted locally in a clinical network, e.g. the one described below, or remotely in a data center.

The proposed computer-automated method operates in the context of a laboratory information system (LIS) which in turn is typically part of a larger clinical network environment, such as a hospital information system (HIS) or picture archiving and communication system (PACS). In the LIS, the image data files will be retained in a database, typically a patient information database containing the electronic medical records of individual patients. The image data files will be taken from stained tissue samples mounted on slides, the slides bearing printed barcode labels by which the Image data files are tagged with suitable metadata, since the microscopes acquiring the Image data files are equipped with barcode readers. From a hardware perspective, the LIS will be a conventional computer network, such as a local area network (LAN) with wired and wireless connections as desired.

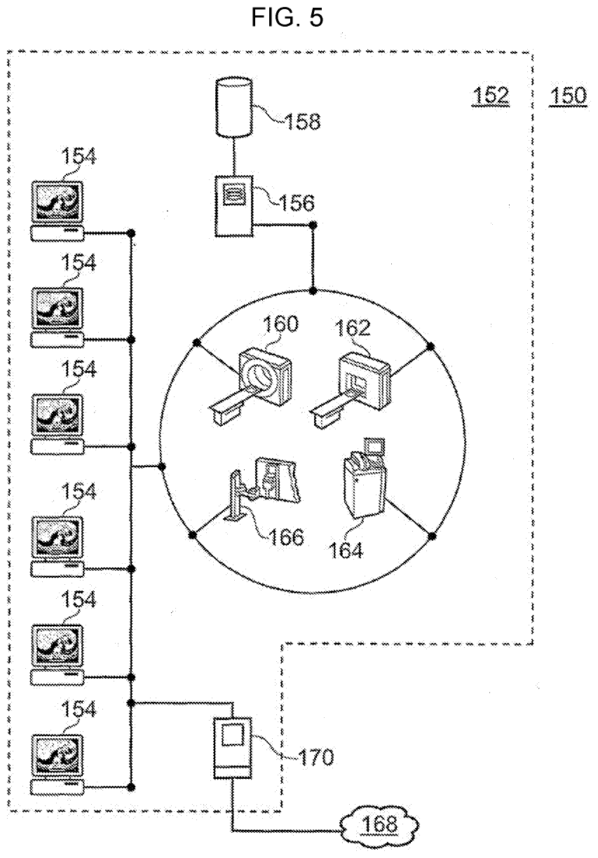

FIG. 5 shows an example computer network which can be used in conjunction with embodiments of the invention. The network 150 comprises a LAN in a hospital 152. The hospital 152 is equipped with a number of workstations 154 which each have access, via the local area network, to a hospital computer server 156 having an associated storage device 158. A LIS, HIS or PACS archive is stored on the storage device 158 so that data in the archive can be accessed from any of the workstations 154. One or more of the workstations 154 has access to a graphics card and to software for computer-implementation of methods of generating images as described hereinbefore. The software may be stored locally at the or each workstation 154, or may be stored remotely and downloaded over the network 150 to a workstation 154 when needed. In other example, methods embodying the invention may be executed on the computer server with the workstations 154 operating as terminals. For example, the workstations may be configured to receive user input defining a desired FISH image data set and to display resulting images while image processing analysis is performed elsewhere in the system. Also, a number of FISH-image acquiring devices and other medical imaging devices 160, 162, 164, 166 are connected to the hospital computer server 156. Image data collected with the devices 160, 162, 164, 166 can be stored directly into the LIS, HIS or PACS archive on the storage device 156. Thus, FISH images can be viewed and processed immediately after the corresponding FISH image data are recorded. The local area network is connected to the Internet 168 by a hospital Internet server 170, which allows remote access to the LIS, HIS or PACS archive. This is of use for remote accessing of the data and for transferring data between hospitals, for example, if a patient is moved, or to allow external research to be undertaken.

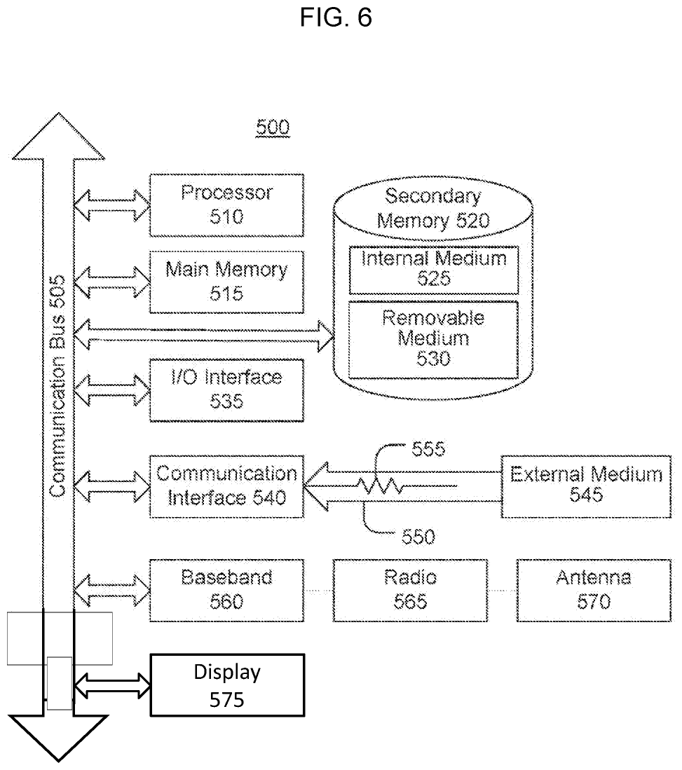

FIG. 6 is a block diagram illustrating an example computing apparatus 500 that may be used in connection with various embodiments described herein. For example, computing apparatus 500 may be used as a computing node in the above-mentioned LIS or PACS system, for example a host computer from which processing of FISH images is carried out in conjunction with a suitable CPU or GPU.

Computing apparatus 500 can be a server or any conventional personal computer, or any other processor-enabled device that is capable of wired or wireless data communication. Other computing apparatus, systems and/or architectures may be also used, including devices that are not capable of wired or wireless data communication, as will be clear to those skilled in the art.

Computing apparatus 500 preferably includes one or more processors, such as processor 510. The processor 510 may be for example a CPU or GPU or arrays or combinations thereof such as CPU and GPU combinations. Additional processors may be provided, such as an auxiliary processor to manage input/output, an auxiliary processor to perform floating point mathematical operations such as a tensor processing unit (TPU), a special-purpose microprocessor having an architecture suitable for fast execution of signal processing algorithms (e.g., digital signal processor, image processor), a slave processor subordinate to the main processing system (e.g., back-end processor), an additional microprocessor or controller for dual or multiple processor systems, or a coprocessor. Such auxiliary processors may be discrete processors or may be integrated with the processor 510. Examples of CPUs which may be used with computing apparatus 500 are, the Pentium processor, Core i7 processor, and Xeon processor, all of which are available from Intel Corporation of Santa Clara, Calif. An example GPU which may be used with computing apparatus 500 is Tesla K80 GPU of Nvidia Corporation, Santa Clara, Calif.

Processor 510 is connected to a communication bus 505. Communication bus 505 may include a data channel for facilitating information transfer between storage and other peripheral components of computing apparatus 500. Communication bus 505 further may provide a set of signals used for communication with processor 510, including a data bus, address bus, and control bus (not shown). Communication bus 505 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, or standards promulgated by the Institute of Electrical and Electronics Engineers (IEEE) including IEEE 488 general-purpose interface bus (GPM), IEEE 696/S-100, and the like.

Computing apparatus 500 preferably includes a main memory 515 and may also include a secondary memory 520. Main memory 515 provides storage of instructions and data for programs executing on processor 510, such as one or more of the functions and/or modules discussed above. It should be understood that computer readable program instructions stored in the memory and executed by processor 510 may be assembler instructions, instruction-set-architecture (ISA) instructions, machine instructions, machine dependent instructions, microcode, firmware instructions, state-setting data, configuration data for integrated circuitry, or either source code or object code written in and/or compiled from any combination of one or more programming languages, including without limitation Smalltalk, C/C++, Java, JavaScript, Perl, Visual Basic, .NET, and the like. Main memory 515 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Secondary memory 520 may optionally include an internal memory 525 and/or a removable medium 530. Removable medium 530 is read from and/or written to in any well-known manner. Removable storage medium 530 may be, for example, a magnetic tape drive, a compact disc (CD) drive, a digital versatile disc (DVD) drive, other optical drive, a flash memory drive, etc.

Removable storage medium 530 is a non-transitory computer-readable medium having stored thereon computer-executable code (i.e., software) and/or data. The computer software or data stored on removable storage medium 530 is read into computing apparatus 500 for execution by processor 510.

The secondary memory 520 may include other similar elements for allowing computer programs or other data or instructions to be loaded into computing apparatus 500. Such means may include, for example, an external storage medium 545 and a communication interface 540, which allows software and data to be transferred from external storage medium 545 to computing apparatus 500. Examples of external storage medium 545 may include an external hard disk drive, an external optical drive, an external magneto-optical drive, etc. Other examples of secondary memory 520 may include semiconductor-based memory such as programmable read-only memory (PROM), erasable programmable read-only memory (EPROM), electrically erasable read-only memory (EEPROM), or flash memory (block-oriented memory similar to EEPROM).

As mentioned above, computing apparatus 500 may include a communication interface 540. Communication interface 540 allows software and data to be transferred between computing apparatus 500 and external devices (e.g. printers), networks, or other information sources. For example, computer software or executable code may be transferred to computing apparatus 500 from a network server via communication interface 540. Examples of communication interface 540 include a built-in network adapter, network interface card (NIC), Personal Computer Memory Card International Association (PCMCIA) network card, card bus network adapter, wireless network adapter, Universal Serial Bus (USB) network adapter, modem, a network interface card (NIC), a wireless data card, a communications port, an infrared interface, an IEEE 1394 fire-wire, or any other device capable of interfacing system 550 with a network or another computing device. Communication interface 540 preferably implements industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software and data transferred via communication interface 540 are generally in the form of electrical communication signals 555. These signals 555 may be provided to communication interface 540 via a communication channel 550. In an embodiment, communication channel 550 may be a wired or wireless network, or any variety of other communication links. Communication channel 550 carries signals 555 and can be implemented using a variety of wired or wireless communication means including wire or cable, fiber optics, conventional phone line, cellular phone link, wireless data communication link, radio frequency (“RF”) link, or infrared link, just to name a few.

Computer-executable code (i.e., computer programs or software) is stored in main memory 515 and/or the secondary memory 520. Computer programs can also be received via communication interface 540 and stored in main memory 515 and/or secondary memory 520. Such computer programs, when executed, enable computing apparatus 500 to perform the various functions of the disclosed embodiments as described elsewhere herein.

In this document, the term “computer-readable medium” is used to refer to any non-transitory computer-readable storage media used to provide computer-executable code (e.g., software and computer programs) to computing apparatus 500. Examples of such media include main memory 515, secondary memory 520 (including internal memory 525, removable medium 530, and external storage medium 545), and any peripheral device communicatively coupled with communication interface 540 (including a network information server or other network device). These non-transitory computer-readable media are means for providing executable code, programming instructions, and software to computing apparatus 500. In an embodiment that is implemented using software, the software may be stored on a computer-readable medium and loaded into computing apparatus 500 by way of removable medium 530, I/O interface 535, or communication interface 540. In such an embodiment, the software is loaded into computing apparatus 500 in the form of electrical communication signals 555. The software, when executed by processor 510, preferably causes processor 510 to perform the features and functions described elsewhere herein.

I/O interface 535 provides an interface between one or more components of computing apparatus 500 and one or more input and/or output devices. Example input devices include, without limitation, keyboards, touch screens or other touch-sensitive devices, biometric sensing devices, computer mice, trackballs, pen-based pointing devices, and the like. Examples of output devices include, without limitation, cathode ray tubes (CRTs), plasma displays, light-emitting diode (LED) displays, liquid crystal displays (LCDs), printers, vacuum florescent displays (VFDs), surface-conduction electron-emitter displays (SEDs), field emission displays (FEDs), and the like.

Computing apparatus 500 also includes optional wireless communication components that facilitate wireless communication over a voice network and/or a data network. The wireless communication components comprise an antenna system 570, a radio system 565, and a baseband system 560. In computing apparatus 500, radio frequency (RF) signals are transmitted and received over the air by antenna system 570 under the management of radio system 565.

Antenna system 570 may comprise one or more antennae and one or more multiplexors (not shown) that perform a switching function to provide antenna system 570 with transmit and receive signal paths. In the receive path, received RF signals can be coupled from a multiplexor to a low noise amplifier (not shown) that amplifies the received RF signal and sends the amplified signal to radio system 565.

Radio system 565 may comprise one or more radios that are configured to communicate over various frequencies. In an embodiment, radio system 565 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from radio system 565 to baseband system 560.

If the received signal contains audio information, then baseband system 560 decodes the signal and converts it to an analog signal. Then the signal is amplified and sent to a speaker. Baseband system 560 also receives analog audio signals from a microphone. These analog audio signals are converted to digital signals and encoded by baseband system 560. Baseband system 560 also codes the digital signals for transmission and generates a baseband transmit audio signal that is routed to the modulator portion of radio system 565. The modulator mixes the baseband transmit audio signal with an RF carrier signal generating an RF transmit signal that is routed to antenna system 570 and may pass through a power amplifier (not shown). The power amplifier amplifies the RF transmit signal and routes it to antenna system 570 where the signal is switched to the antenna port for transmission.

Baseband system 560 is also communicatively coupled with processor 510, which may be a CPU. Processor 510 has access to data storage areas 515 and 520. Processor 510 is preferably configured to execute instructions (i.e., computer programs or software) that can be stored in main memory 515 or secondary memory 520. Computer programs can also be received from baseband processor 560 and stored in main memory 510 or in secondary memory 520, or executed upon receipt. Such computer programs, when executed, enable computing apparatus 500 to perform the various functions of the disclosed embodiments. For example, data storage areas 515 or 520 may include various software modules.

The computing apparatus further comprises a display 575 directly attached to the communication bus 505 which may be provided instead of or addition to any display connected to the I/O interface 535 referred to above.

Various embodiments may also be implemented primarily in hardware using, for example, components such as application specific integrated circuits (ASICs), programmable logic arrays (PLA), or field programmable gate arrays (FPGAs). Implementation of a hardware state machine capable of performing the functions described herein will also be apparent to those skilled in the relevant art. Various embodiments may also be implemented using a combination of both hardware and software.

Furthermore, those of skill in the art will appreciate that the various illustrative logical blocks, modules, circuits, and method steps described in connection with the above described figures and the embodiments disclosed herein can often be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a module, block, circuit, or step is for ease of description. Specific functions or steps can be moved from one module, block, or circuit to another without departing from the invention.

Moreover, the various illustrative logical blocks, modules, functions, and methods described in connection with the embodiments disclosed herein can be implemented or performed with a general purpose processor, a digital signal processor (DSP), an ASIC, FPGA, or other programmable logic device, discrete gate or transistor logic, discrete hardware components, or any combination thereof designed to perform the functions described herein. A general-purpose processor can be a microprocessor, but in the alternative, the processor can be any processor, controller, microcontroller, or state machine. A processor can also be implemented as a combination of computing devices, for example, a combination of a DSP and a microprocessor, a plurality of microprocessors, one or more microprocessors in conjunction with a DSP core, or any other such configuration.

Additionally, the steps of a method or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of storage medium including a network storage medium. An exemplary storage medium can be coupled to the processor such that the processor can read information from, and write information to, the storage medium. In the alternative, the storage medium can be integral to the processor. The processor and the storage medium can also reside in an ASIC.

A computer readable storage medium, as referred to herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Any of the software components described herein may take a variety of forms. For example, a component may be a stand-alone software package, or it may be a software package incorporated as a “tool” in a larger software product. It may be downloadable from a network, for example, a website, as a stand-alone product or as an add-in package for installation in an existing software application. It may also be available as a client-server software application, as a web-enabled software application, and/or as a mobile application.

Embodiments of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

The computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The illustrated flowcharts and block diagrams illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It will be clear to one skilled in the art that many improvements and modifications can be made to the foregoing exemplary embodiment without departing from the scope of the present disclosure.

1.-14. (canceled)

15. An image acquisition apparatus for acquiring an image of a tissue sample, the apparatus comprising:
a microscope operable to acquire images of the tissue sample; and
at least one hardware processor configured to set an exposure for acquiring an image of the tissue sample to an exposure value,
wherein the exposure value is set to acquire an underexposed image,
acquire the image with the exposure value,
apply at least one morphological operator to identify areas of interest around locations of elevated image intensity,
generate an image mask for filtering out all but the areas of interest,
apply the mask to the image to determine image intensity in the areas of interest,
determine if the image intensity has an intensity value that indicates correct exposure of the image,
when the image has the correct exposure, save the image, and
when the image does not have the correct exposure,
adjust the exposure value,
acquire a new image with the adjusted exposure value,
apply the mask to the new image to determine image intensity in the areas of interest, and
determine if the image intensity of the new image has an intensity value that indicates correct exposure of the new image.

16. The apparatus of claim 15, wherein the tissue sample comprises a fluorescent in situ hybridization (FISH) probe that is sensitive to a fluorescent label used to tag deoxyribonucleic acid (DNA) or ribonucleic acid (RNA).

17. The apparatus of claim 15, wherein the at least one hardware processor is configured to iteratively, until an image intensity of an acquired image indicates correct exposure of the acquired image or a maximum exposure value is reached:
adjust the exposure value;
acquire an image with the adjusted exposure value;
apply the mask to the acquired image to determine image intensity in the areas of interest; and
determine if the image intensity of the acquired image has an intensity value that indicates correct exposure of the acquired image.

18. The apparatus of claim 17, wherein the at least one hardware processor is configured to, when a maximum exposure value is reached before the image intensity of the acquired image indicates correct exposure, save a most recently acquired image.

19. The apparatus of claim 17, wherein adjusting the exposure value comprises increasing the exposure value by an increment.

20. The apparatus of claim 19, wherein the increment is a fixed amount across every iteration.

21. The apparatus of claim 19, wherein the increment varies across iterations.

22. The apparatus of claim 21, wherein a size of the increment decreases in successive iterations.

23. The apparatus of claim 17, wherein adjusting the exposure value comprises multiplying a first factor, based on a sum of ratios between the exposure values and the image intensities of the acquired images in each iteration, with a second factor based on a target intensity value that indicates correct exposure.

24. The apparatus of claim 17, wherein adjusting the exposure value comprises multiplying a first factor, based on a ratio between the exposure value and the image intensity of the acquired image in one iteration, with a second factor based on a target intensity value that indicates correct exposure.

25. The apparatus of claim 15, wherein adjusting the exposure value comprises multiplying a first factor, based on a ratio between the exposure value and the image intensity of the image, with a second factor based on a target intensity value that indicates correct exposure.

26. The apparatus of claim 15, wherein an intensity value indicates correct exposure when the intensity value equals or exceeds a target intensity value.

27. The apparatus of claim 15, wherein the at least one hardware processor is further configured to, prior to acquiring the image with the exposure value:
acquire a counterstain image of the tissue sample;
generate a counterstain image mask that filters out all but areas of or around counterstained areas; and
confine application of the at least one morphological operator to the areas of or around the counterstained areas that are not filtered out by the counterstain image mask.

28. The apparatus of claim 27, wherein confining application of the at least one morphological operator to the areas of or around the counterstained areas that are not filtered out by the counterstain image mask comprises setting an intensity value of all pixels within the counterstain image mask to zero.

29. The apparatus of claim 15, wherein the at least one morphological operator comprises a regional maximum operator that identifies regional maxima within the image.

30. The apparatus of claim 29, wherein each of the regional maxima is defined as a contiguous set of connected pixels that have intensity values above a threshold and whose external boundary pixels have intensity values below the threshold.

31. The apparatus of claim 29, wherein the at least one morphological operator further comprises a dilation operator that dilates each of the regional maxima.

32. The apparatus of claim 29, wherein the mask filters out areas that are not within the identified regional maxima.

33. A non-transitory computer-readable medium having instructions stored thereon, wherein the instructions, when executed by a processor, cause the processor to:
set an exposure for acquiring an image of a tissue sample to an exposure value, wherein the exposure value is set to acquire an underexposed image;
acquire the image with the exposure value via a microscope;
apply at least one morphological operator to identify areas of interest around locations of elevated image intensity;
generate an image mask for filtering out all but the areas of interest;
apply the mask to the image to determine image intensity in the areas of interest;
determine if the image intensity has an intensity value that indicates correct exposure of the image;
when the image has the correct exposure, save the image; and
when the image does not have the correct exposure;
adjust the exposure value,
acquire a new image with the adjusted exposure value,
apply the mask to the new image to determine image intensity in the areas of interest, and
determine if the image intensity of the new image has an intensity value that indicates correct exposure of the new image.

34. A method of acquiring an image of a tissue sample using at least one hardware processor, the method comprising:
setting an exposure for acquiring an image of the tissue sample to an exposure value, wherein the exposure value is set to acquire an underexposed image;
acquiring the image with the exposure value via a microscope;
applying at least one morphological operator to identify areas of interest around locations of elevated image intensity;
generating an image mask for filtering out all but the areas of interest;
applying the mask to the image to determine image intensity in the areas of interest;
determining if the image intensity has an intensity value that indicates correct exposure of the image;
when the image has the correct exposure, saving the image; and
when the image does not have the correct exposure;
adjusting the exposure value,
acquiring a new image with the adjusted exposure value,
applying the mask to the new image to determine image intensity in the areas of interest, and
determining if the image intensity of the new image has an intensity value that indicates correct exposure of the new image.
